(19) United States
(12) Patent Application Publication
(10) Pub. No.: US 2023/0009654 A1
(43) Pub. Date: Jan. 12, 2023

(54) EXCREMENT MANAGEMENT SYSTEM, EXCRETION INFORMATION MANAGEMENT METHOD, COMPUTER PROGRAM, EDGE SERVER, AND TOILET SEAT DEVICE

(71) Applicant: TOTO LTD., Kitakyushu-shi, Fukuoka (JP)

(72) Inventors: Takeshi Takaki, Fukuoka (JP); Satoko Kizuka, Fukuoka (JP); Yuta Sakai, Fukuoka (JP); Hirotomo Suyama, Fukuoka (JP); Hitoaki Higuchi, Fukuoka (JP); Haruna Matsui, Fukuoka (JP); Tetsuhiro Wasada, Fukuoka (JP); Ryo Oi, Fukuoka (JP)

(21) Appl. No.: 17/277,862

(22) PCT Filed: Aug. 31, 2020
(86) PCT No.: PCT/JP2020/032904

§ 371 Date: Mar. 19, 2021

(30) Foreign Application Priority Data
Apr. 3, 2020 (JP) 2020-067715
Aug. 25, 2020 (JP) 2020-141388

(51) Int. Cl. G01N 33/483; E03D 9/00; G01N 21/27; G16H 40/63

(52) U.S. Cl. CPC G01N 33/4833; E03D 9/00; G01N 21/27; G16H 40/63; H04L 67/12

(57) ABSTRACT

An excrement management system according to an embodiment includes: a closet bowl in which a bowl part for receiving excrement is formed; a light emitting unit configured to emit light toward an inner part of the closet bowl; a light receiving unit comprising an image sensor configured to receive light; a cloud server and an edge server configured to analyze light reception data received by the light receiving unit; a first communication device configured to transmit the light reception data to the cloud server; and a second communication device configured to transmit the light reception data to the edge server. The cloud server analyzes the light reception data to determine a characteristic of excrement, and the edge server analyzes the light reception data to determine whether to transmit the light reception data to the cloud server by the first communication device.

### FIELD

An embodiment of the present disclosure relates to an excrement management system, an excretion information management method, a computer program, an edge server, and a toilet seat device.

### BACKGROUND

Conventionally, in nursing homes and the like, nursing staffs record bowel movement conditions of inmates to manage states of health of the inmates. As a device that can reduce a load of recording bowel movement conditions, there is known a device for estimating a state of health of a human body by photographing excrement excreted in a toilet as an image to analyze the image (for example, refer to Patent Literature 1).

The data detection device disclosed in Patent Literature 1 includes an image photographing unit that photographs an image of excrement and a data processing/analyzing unit that analyzes a color and a shape of the excrement based on the photographed image, and the data processing/analyzing unit associates the color and the shape that are analyzed based on the image of the excrement with a state of health to estimate the state of health of a human body.

### CITATION LIST

### Patent Literature

Patent Literature 1: Japanese Patent Application Laid-open No. 2007-252805

### SUMMARY

### Technical Problem

To analyze the color and the shape based on the image of the excrement, it is preferable to use a cloud server including abundant calculation resources and storages. On the other hand, a data amount of image data is relatively large, so that there is the problem that a data communication amount is largely increased when all pieces of image data photographed by the image photographing unit are transmitted to the cloud server.

To solve the problem described above, the embodiment of the present disclosure provides an excrement management system, an excretion information management method, a computer program, an edge server, and a toilet seat device that can suppress the data communication amount to the cloud server that analyzes image data of excrement.

### Solution to Problem

An excrement management system according to an aspect of the embodiment, configured to collect and manage information about excrement, the excrement management system comprising: a closet bowl in which a bowl part for receiving excrement is formed; a light emitting unit configured to emit light toward an inner part of the closet bowl; a light receiving unit comprising an image sensor configured to receive light; a cloud server and an edge server configured to analyze light reception data received by the light receiving unit; a first communication device configured to transmit the light reception data to the cloud server; and a second communication device configured to transmit the light reception data to the edge server, wherein the cloud server analyzes the light reception data to determine a characteristic of excrement, and the edge server analyzes the light reception data to determine whether to transmit the light reception data to the cloud server by the first communication device.

With the excrement management system according to an aspect of the embodiment, it is possible to determine whether the light reception data should be transmitted to the cloud server by the first communication device by analyzing the light reception data by the edge server before the light reception data received by the light receiving unit is transmitted to the cloud server by the first communication device. Due to this, it is possible to prevent all pieces of the light reception data received by the light receiving unit from being entirely transmitted to the cloud server, so that the data communication amount to the cloud server can be suppressed.

In the excrement management system according to an aspect of the embodiment, the first communication device transmits the light reception data to the cloud server by using a wide-area information communication network, and the second communication device transmits the light reception data to the edge server by using a local-area information communication network.

With the excrement management system according to an aspect of the embodiment, communication to the cloud server is performed by using a wide-area information communication network. Thus, a degree of freedom of an installation position can be secured for the cloud server that requires a space because of abundant calculation resources and storages included therein. With the excrement management system according to an aspect of the embodiment, communication to the edge server is performed by using a local-area information communication network. Thus, the data received by the light receiving unit can be transmitted to the edge server via the local-area information communication network that can perform data communication without communication utility charge. Due to this, it is possible to reduce communication utility charge for data communication for transmitting the light reception data to the edge server.

In the excrement management system according to an aspect of the embodiment, the first communication device is configured to perform transmission/reception of data between the cloud server and the edge server, and data capacity that is transmitted from the cloud server to the edge server via the first communication device is smaller than data capacity of the light reception data that is transmitted from the edge server to the cloud server via the first communication device.

The data transmitted to the cloud server is a large capacity of light reception data including sufficient information required for analyzing a characteristic of the excrement by the cloud server. On the other hand, the data transmitted from the cloud server is only an analysis result related to the characteristic of the excrement, so that the data transmitted from the cloud server is not required to be a large capacity of light reception data.

On the other hand, the excrement management system according to an aspect of the embodiment is configured such that the first communication device performs data transmission/reception between the cloud server and the edge server, and the capacity of data transmitted from the cloud server to the edge server is smaller than the capacity of data transmitted from the edge server to the cloud server in data transmission/reception between the cloud server and the edge server. Due to this, the data communication amount can be suppressed in data transmission/reception between the cloud server and the edge server.

In the excrement management system according to an aspect of the embodiment, the cloud server analyzes the light reception data to determine at least one of three characteristic amounts including a color, a shape, and an amount of excrement, and the edge server analyzes the light reception data to determine whether excrement is included in the light reception data.

In the excrement management system according to an aspect of the embodiment, the cloud server determines at least one of three characteristic amounts including the color, the shape, and the amount of the excrement, and the edge server determines whether the excrement is included in the light reception data. Due to this, analysis of the light reception data by the edge server is enabled to be simplified as compared with analysis of the light reception data by the cloud server, and the edge server having a calculation resource smaller than that of the cloud server can be efficiently used. Thus, it is possible to suppress communication delay caused by interruption due to determination performed by the edge server before transmitting the light reception data to the cloud server.

The excrement management system according to an aspect of the embodiment further includes: a user identification device configured to acquire user information who uses the closet bowl, wherein the first communication device does not transmit the user information to the cloud server.

The excrement management system according to an aspect of the embodiment includes the user identification device that acquires the user information, so that the characteristic of the excrement determined by the cloud server can be associated with the user information about the user who has excreted the excrement. The first communication device does not transmit the user information to the cloud server, so that the user information is excluded from the light reception data transmitted from the edge server. Accordingly, communication capacity from the edge server to the cloud server can be reduced. The data transmitted from the edge server to the cloud server does not include information with which an individual can be specified, so that individual information can be prevented from leaking out in a communication path.

In the present embodiment, the “user information” means information with which an individual can be specified (for example, a name or an address), and does not include information that is anonymized by the edge server and the like so that an individual cannot be specified (an ID and the like).

In the excrement management system according to an aspect of the embodiment, user information about a user who uses the closet bowl is previously recorded in the cloud server, and the light reception data that is determined to be transmitted by the edge server is associated with the user information that is previously recorded in the cloud server.

With the excrement management system according to an aspect of the embodiment, the light reception data and the characteristic information about the excrement analyzed by the cloud server can be transmitted to an information terminal of a user, a medical institution, and the like by associating the user information with the light reception data in the cloud server, so that usability of the light reception data and the characteristic information about the excrement can be enhanced.

An excretion information management method according to an aspect of the embodiment, for managing, on a cloud server, information about excrement collected in a toilet room in which a closet bowl is disposed, the excretion information management method comprising: a detection step of receiving, by a light receiving unit, reflected light from excrement corresponding to light that is emitted toward an inner part of the closet bowl by a light emitting unit; and an analysis step of determining whether to transmit the light reception data to the cloud server by a communication device based on light reception data detected at the detection step.

With the excretion information management method according to an aspect of the embodiment, in a case of receiving reflected light from the excrement corresponding to light that is emitted by the light emitting unit toward an inner part of a closet bowl, it is determined whether to transmit the light reception data to the cloud server by the communication device by analyzing the light reception data. Due to this, in a case in which the light reception data is data not deserving to be managed on the cloud server, it is possible to prevent the communication device from transmitting the light reception data to the cloud server. Due to this, with the excretion information management method according to an aspect of the embodiment, the data communication amount to the cloud server can be suppressed.

A computer program according to an aspect of the embodiment, executed by an edge server configured to be able to communicate with a closet bowl device and a cloud server, the computer program configured to cause an edge server to perform: a reception procedure of receiving light reception data that is detected by receiving, by a light receiving unit, reflected light from excrement corresponding to light that is emitted toward an inner part of a closet bowl by a light emitting unit; and a transmission procedure of transmitting a determination result about whether to transmit the light reception data to a device configured to control a communication device that transmits the light reception data to the cloud server based on an analysis result about the light reception data.

With the computer program according to an aspect of the embodiment, by analyzing the light reception data received through the reception procedure and transmitting a determination result about whether to transmit the light reception data to the device for controlling the communication device that transmits the light reception data to the cloud server, the data communication amount to the cloud server can be suppressed.

An edge server according to an aspect of the embodiment, configured to be able to communicate with a cloud server and a closet bowl device, the edge server comprising: a first communication device configured to be able to communicate with the cloud server; a second communication device configured to be able to communicate with the closet bowl device; a memory configured to store detection data related to excrement that is optically detected and transmitted from the closet bowl device via the second communication device; and an arithmetic processing device configured to analyze the detection data stored in the memory, wherein the arithmetic processing device determines whether to transmit the detection data to the cloud server by the first communication device based on the detection data.

The edge server according to an aspect of the embodiment analyzes the detection data related to the excrement transmitted from the closet bowl device, and determines whether to transmit the detection data to the cloud server by the first communication device. Due to this, it is possible to prevent all pieces of the detection data transmitted from the closet bowl device from being entirely transmitted to the cloud server, so that the data communication amount to the cloud server can be suppressed.

A toilet seat device according to an aspect of the embodiment, disposed on an upper part of a closet bowl, the toilet seat device comprising: a light emitting unit configured to emit light toward an inner part of the closet bowl; a light receiving unit comprising an image sensor configured to receive light; a memory configured to store light reception data received by the light receiving unit; a communication device configured to transmit the light reception data to a cloud server; and an arithmetic processing device configured to analyze the light reception data stored in the memory, wherein the arithmetic processing device determines whether to transmit the light reception data to the cloud server based on the light reception data.

The toilet seat device according to an aspect of the embodiment analyzes the light reception data received by the light receiving unit disposed on the toilet seat device, and determines whether to transmit the light reception data to the cloud server by the communication device. Due to this, it is possible to prevent all pieces of the light reception data received by the light receiving unit from being entirely transmitted to the cloud server, so that the data communication amount to the cloud server can be suppressed.

### Advantageous Effects of Invention

According to an aspect of the embodiment, it is possible to suppress a data communication amount to a cloud server that analyzes image data of excrement.

### BRIEF DESCRIPTION OF DRAWINGS

FIG. 1 is a perspective view illustrating an example of a configuration of an excrement management system according to an embodiment of the present invention.

FIG. 2 is an external appearance perspective view of a detection device according to the embodiment of the present invention.

FIG. 3 is a block diagram illustrating a functional configuration of the excrement management system according to the embodiment of the present invention.

FIG. 4 is a block diagram illustrating an example of the functional configuration of the excrement management system according to the embodiment of the present invention.

FIG. 5 is a conceptual diagram corresponding to the block diagram of the functional configuration in FIG. 4.

FIG. 6 is a block diagram illustrating an example of the functional configuration of the excrement management system according to the embodiment of the present invention.

FIG. 7 is a conceptual diagram corresponding to the block diagram of the functional configuration in FIG. 6.

FIG. 8 is a block diagram illustrating an example of the functional configuration of the excrement management system according to the embodiment of the present invention.

FIG. 9 is a conceptual diagram corresponding to the block diagram of the functional configuration in FIG. 8.

FIG. 10 is a block diagram illustrating an example of data in processing of the excrement management system according to the embodiment of the present invention.

FIG. 11 is a diagram illustrating an example of data analysis of a shape and an amount of excrement.

FIG. 12 is a diagram illustrating an example of data analysis of a color of excrement.

FIG. 13 is a diagram illustrating an example of a relation between excrement and blood.

FIG. 14 is a diagram illustrating an example of data analysis of a color of excrement.

FIG. 15 is a diagram illustrating an example of data analysis of a color of excrement.

### DESCRIPTION OF EMBODIMENTS

The following describes an embodiment of an excrement management system disclosed herein in detail with reference to the attached drawings. The present invention is not limited to the embodiment described below. The following describes a configuration and information processing for collecting and managing information about excrement excreted by a user of a toilet room.

1. External Appearance Configuration of Toilet Room

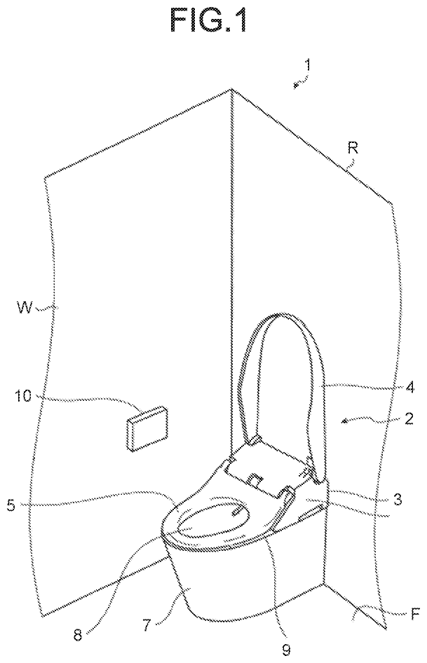

First, the following describes an external appearance configuration of a toilet room according to an embodiment of the present invention with reference to FIG. 1. FIG. 1 is a schematic perspective view illustrating an example of the external appearance configuration of the toilet room according to the embodiment.

As illustrated in FIG. 1, an excrement management system 1 includes a closet bowl device 2 and an operation device 10. As illustrated in FIG. 1, in a toilet room R, a Western-style closet bowl (hereinafter, referred to as a “toilet bowl”) 7 is disposed on a floor face F. Hereinafter, a direction from the floor face F toward an inside space of the toilet room R is referred to as an upper direction. A toilet seat device 3 is disposed on an upper part of the toilet bowl 7.

The toilet bowl 7 is, for example, made of ceramic. A bowl part 8 is formed on the toilet bowl 7. The bowl part 8 is a portion that has a shape depressed downward, and receives excrement of a user. The toilet bowl 7 is not limited to a floor-standing type as illustrated in the drawing, and may be a wall-hanging type and the like. A rim part 9 is disposed over the entire circumference of an end part of an opening that the bowl part 8 faces in the toilet bowl 7. A washing water tank for storing washing water may be installed in the toilet bowl 7, or the toilet bowl 7 may be what is called a tankless type, which does not include the washing water tank.

For example, when the user operates a washing operation unit (not illustrated) for washing disposed in the toilet room R, the toilet bowl is washed by supplying washing water to the bowl part 8 of the toilet bowl 7. The washing operation unit may be an operation lever or a pressing operation on a button for washing the toilet bowl disposed on the operation device 10. The washing operation unit is not limited to the operation lever and the like that cause the toilet bowl to be washed by being manually operated by the user, and may be a seating sensor that causes the toilet bowl to be washed by detecting a human body with a sensor for detecting the user.

The toilet seat device 3 is attached to an upper part of the toilet bowl 7, and includes a toilet lid 4, a toilet seat 5, and a functional unit 6. The toilet seat device 3 may be attached to the toilet bowl 7 in a detachable manner, or may be integrally attached to the toilet bowl 7.

As illustrated in FIG. 1, the toilet seat 5 is formed in a ring shape having a center opening, and disposed at a position overlapping with the opening of the toilet bowl 7 along the rim part 9. The toilet seat 5 functions as a seating part that supports buttocks of the seated user. As illustrated in FIG. 1, the toilet lid 4 and the toilet seat 5 are attached so that respective end parts thereof are pivotally supported by the functional unit 6, and enabled to rotate (enabled to be opened and closed) about a pivotally supporting portion of the functional unit 6. The toilet lid 4 is attached to the toilet seat device 3 as needed, and the toilet seat device 3 does not necessarily include the toilet lid 4.

The operation device 10 is disposed in the toilet room R. The operation device 10 is disposed at a position that can be operated by the user at the time when the user is seated on the toilet seat 5. In the example illustrated in FIG. 1, the operation device 10 is disposed on a wall face W on the right side of the user seated on the toilet seat 5. The operation device 10 is not necessarily disposed on the wall face, and may be disposed in various modes so long as the user seated on the toilet seat 5 can use the operation device 10. For example, the operation device 10 may be disposed integrally with the toilet seat device 3.

The operation device 10 is connected to be able to communicate with the toilet seat device 3 in a wired or wireless manner via a predetermined network. For example, the toilet seat device 3 may be connected to the operation device 10 in any manner so long as information can be transmitted and received. The toilet seat device 3 and the operation device 10 may be connected to be able to perform wired communication, or may be connected to be able to perform wireless communication.

The excrement management system 1 may identify the user based on an operation performed by the user on the operation device 10. The operation device 10 may also function as a user identification device 38 (refer to FIG. 3) for identifying the user. For example, the operation device 10 identifies the user by personal identification. The operation device 10 may also identify the user based on biological information such as a fingerprint or a vein of the user. In this case, the excrement management system 1 may include the operation device 10 functioning as the user identification device 38. The operation device 10 is not necessarily disposed in the toilet room R so long as a configuration with which the user excretes excrement (the toilet bowl 7, the toilet seat device 3, and the like) is disposed.

2. Functional Configuration of Detection Device

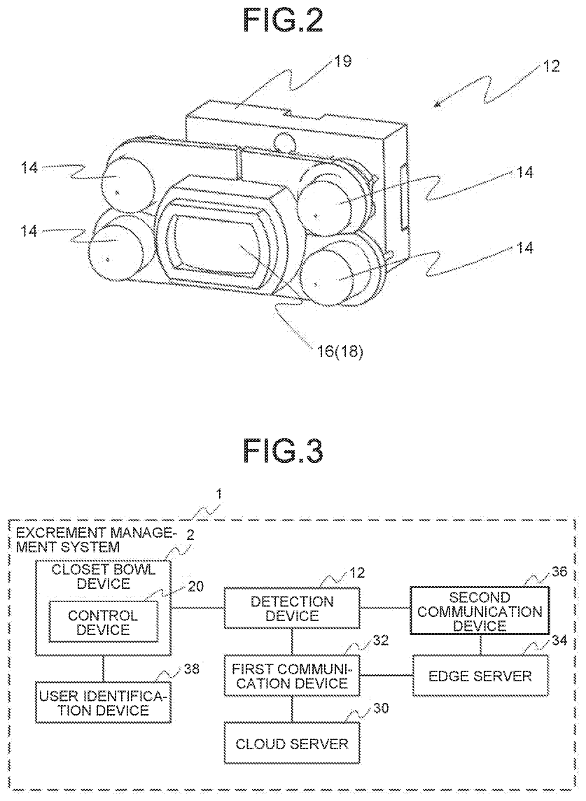

Next, the following describes a functional configuration of a detection device with reference to FIG. 2. FIG. 2 is an external appearance perspective view of the detection device according to the embodiment.

A detection device 12 includes light emitting units 14 that emit light in response to an electric signal controlled by a control device 20 (refer to FIG. 3), a light receiving unit 16 that receives reflected light from excrement of the user corresponding to the light emitted by the light emitting unit 14, and a housing 18 for supporting the light emitting unit 14 and the light receiving unit 16. With this configuration, the detection device 12 implements a function of detecting excrement excreted into the toilet bowl. For example, the detection device 12 detects data including three characteristic amounts including a color, a shape, and an amount of feces excreted by the user. Processing for the data detected by the detection device 12 will be described later.

The light emitting unit 14 includes, for example, a light emitting element (not illustrated) such as a light emitting diode (LED). The light emitting element included in the light emitting unit 14 is not limited to the LED, and various elements may be used. The light emitted from the light emitting unit 14 is not limited to white light having uniform wavelengths of visible rays, and may be colored light having only a specific wavelength, or invisible light such as infrared rays.

The light receiving unit 16 includes a lens 17 and a light receiving element (not illustrated). The light receiving element is, for example, formed of a charge coupled device (CCD) sensor, or a line sensor or an area sensor in which complementary metal oxide semiconductor (CMOS) sensors are arranged. The light receiving unit may have a configuration having a spectral function such as a spectral filter.

The detection device 12 may be disposed in an inner part of the functional unit 6 or the toilet seat 5 included in the toilet seat device 3 to be formed integrally with the toilet seat device 3, or may be hung between the rim part 9 of the toilet bowl 7 and the toilet seat 5 to be formed separately from the toilet seat device 3.

3. Configuration of Excrement Management System

The following describes a configuration of the excrement management system 1 with reference to FIG. 3 to FIG. 9. First, the following describes a functional configuration of the excrement management system 1 with reference to FIG. 3. FIG. 3 is a block diagram illustrating the functional configuration of the excrement management system according to the embodiment.

As illustrated in FIG. 3, the excrement management system 1 includes the closet bowl device 2, the detection device 12, a cloud server 30, a first communication device 32, an edge server 34, a second communication device 36, and a user identification device 38. FIG. 3 illustrates the configuration including separated devices corresponding to respective functions of the excrement management system 1, but the excrement management system 1 may be constituted of a device that implements a plurality of functions. Details of this point will be described later.

The closet bowl device 2 includes the toilet bowl 7 and the toilet seat device 3 in FIG. 1, and is used as a device with which the user excretes feces.

The toilet seat device 3 included in the closet bowl device 2 functions as the control device 20 for controlling the detection device 12. In a case in which a function of the edge server 34 (described later) is executed by the closet bowl device 2, the control device 20 also functions as the edge server 34.

The control device 20 includes an arithmetic processing device 24 for controlling the detection device 12 and performing arithmetic processing on data detected by the detection device 12, and a memory 22 for storing the data detected by the detection device 12 and storing a control program to be executed by the arithmetic processing device 24.

The arithmetic processing device 24 may be implemented, for example, by various units such as a processor including a central processing unit (CPU), a micro processing unit (MPU), an application specific integrated circuit (ASIC), and the like, or an integrated circuit including a field programmable gate array (FPGA) and the like.

The memory 22 includes, for example, various configurations such as a read only memory (ROM) and a random access memory (RAM).

The detection device 12 includes the light emitting unit 14 and the light receiving unit 16 in FIG. 2, and is used as a device that optically detects feces excreted by the user.

The cloud server 30 implements a function of determining a characteristic of excrement based on light reception data detected by the detection device 12. The cloud server 30 may also implement a function of accumulating the detection data detected by the detection device 12 and information about a determination result determined by the cloud server 30 to provide the information in accordance with a demand from the outside.

The cloud server 30 is a server disposed in a cloud environment, which is a virtual server in which arithmetic performance and capacity of a storage (storage device) are scalable. In a case in which the cloud server 30 is connected to a portable terminal (a smartphone or a personal computer) of the user, a form of a web service (for example, an Application Service Provider (ASP)) and the like may be employed as a method of providing information to the user by the excrement management system 1, the web service of performing arithmetic processing based on accumulated data and transmitting a result thereof to the portable terminal via a wide-area information communication network. In this case, the cloud server 30 performs data accumulation, arithmetic processing, response processing for a request for information, and the like.

The first communication device 32 implements a function of transmitting the data detected by the detection device 12 to the cloud server 30. The first communication device 32 is constituted of a wide-area information communication network, what is called a Wide Area Network (WAN), and performs transmission/reception of data between the cloud server 30 and the edge server 34. The wide-area information communication network may be, for example, a wide-area wired communication line such as the Internet and a dedicated line, or a wide-area wireless communication line such as a third-generation mobile communication system (3G), 4G, 5G, LTE, or the like.

The edge server 34 implements a function of determining whether to transmit detection data to the cloud server 30 via the first communication device 32. The edge server 34 is a server that communicates with the cloud server 30 via a wide-area information communication network, and communicates with the control device 20 included in the closet bowl device 2 via a local-area information communication network. Processing for determining whether to transmit the detection data to the cloud server 30 implemented by the edge server 34 will be described later.

The second communication device 36 implements a function of transmitting the data detected by the detection device 12 to the edge server 34. The second communication device 36 is constituted of a local-area information communication network, what is called a Local Area Network (LAN), short-range wireless communication, serial communication, and the like, and performs transmission/reception of data between the edge server 34 and the closet bowl device 2, or between the edge server 34 and a user's portable terminal.

The local-area information communication network may be, for example, a local wired communication line such as a field bus including Profibus, Modbus, TC-net, and the like, and Ethernet (registered trademark), or may be a local wireless communication line such as a wireless LAN (Wi-Fi) (registered trademark) and a 920 MHz band.

The short-range wireless communication may be, for example, classic Bluetooth, or may be Bluetooth Low Energy (BLE) or ZigBee that can implement communication with low power consumption.

The serial communication may be, for example, UART communication, or may be a communication scheme such as I2C communication or SPI communication.

The user identification device 38 implements a function of identifying the user who uses the toilet bowl 7. The user identification device 38 may identify the user by using biological information such as a fingerprint and a vein of the user acquired by various sensors disposed in the operation device 10. Alternatively, the user identification device 38 may specify the user by using information for identifying the user (a user ID and the like) acquired from a communication device through communication between the portable terminal of the user and various communication devices.

The user information acquired by the user identification device 38 is information with which an individual can be specified, so that it is preferable to provide a security measure. Thus, the user information is preferably converted into information that is anonymized by the edge server 34 and the like so that an individual cannot be specified (an ID and the like). Due to this, the first communication device 32 can be prevented from transmitting the information with which an individual can be specified to the cloud server 30. Accordingly, the user information is excluded from the light reception data transmitted from the edge server, so that communication capacity from the edge server 34 to the cloud server 30 can be reduced. The data transmitted from the edge server 34 to the cloud server 30 does not include the information with which an individual can be specified, so that the individual information can be prevented from leaking out in a communication path.

On the other hand, by associating the user information with the light reception data in the cloud server 30, usability of the light reception data and a processing result stored in the cloud server 30 can be enhanced. In this case, by causing the cloud server 30 to previously record the user information with which an individual can be specified that is associated with the anonymized user information transmitted from the edge server 34 to the cloud server 30, for example, the light reception data transmitted from the edge server can be associated with the user information with which an individual can be specified that is recorded in the cloud server 30.

According to this aspect, the light reception data and the processing result stored in the cloud server 30 may be transmitted to the user after being compared with the user information with which an individual can be specified by the edge server 34, or may be transmitted to the user after associating the stored light reception data and the processing result with the user information with which an individual can be specified by the cloud server 30. Furthermore, the light reception data and the processing result stored in the cloud server 30 may be associated with the user information with which an individual can be specified, and transmitted to a medical institution to be used for a diagnosis and the like made by a doctor. Accordingly, usability of the detection data and the processing result stored in the cloud server 30 can be enhanced.

3-1. Configuration Example of Excrement Management System

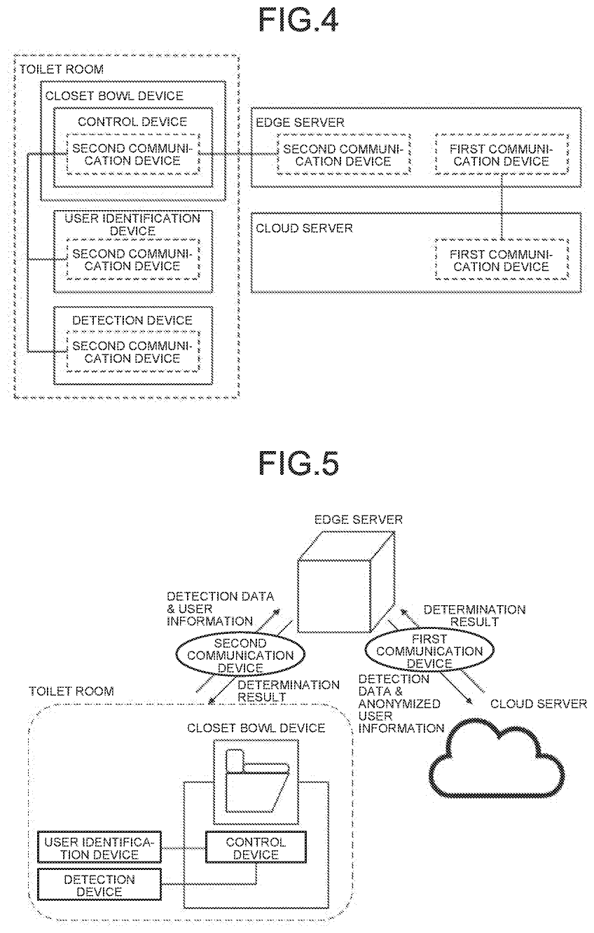

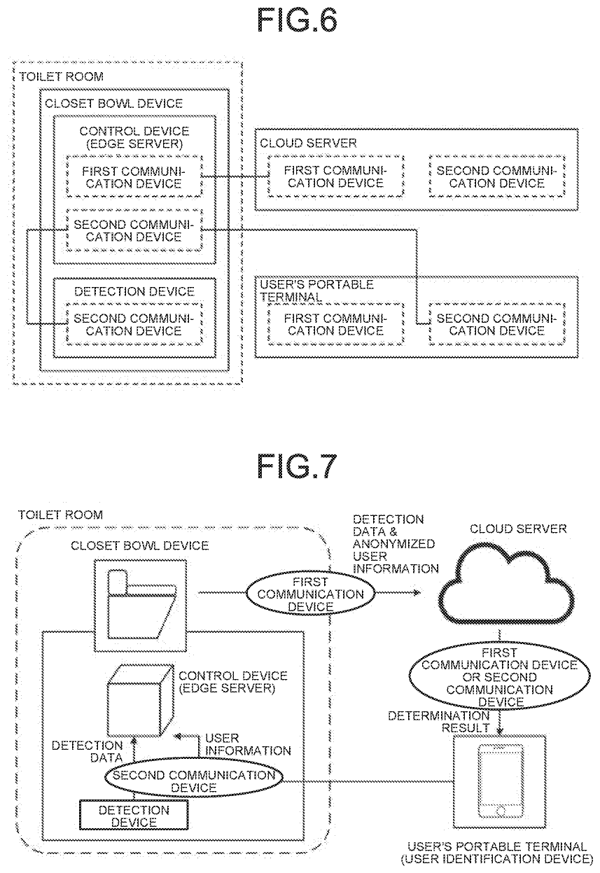

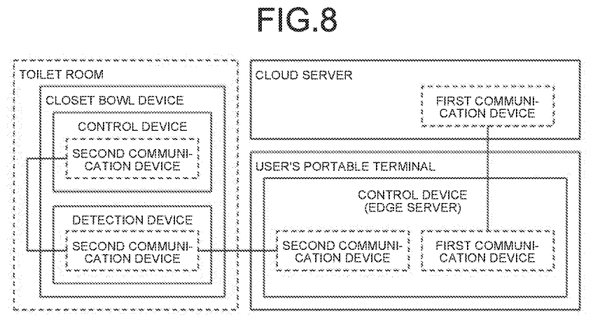

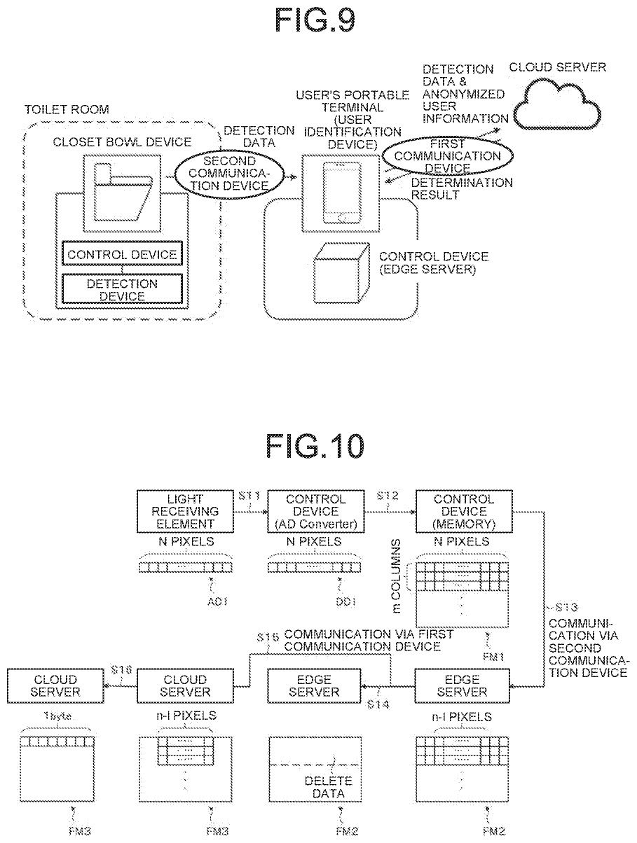

Next, the following describes a configuration example of the excrement management system 1 with reference to FIG. 4 to FIG. 9. FIG. 4, FIG. 6, and FIG. 8 are block diagrams illustrating examples of a functional configuration of the excrement management system according to the embodiment. FIG. 5 is a conceptual diagram corresponding to the block diagram of the functional configuration in FIG. 4. FIG. 7 is a conceptual diagram corresponding to the functional configuration in FIG. 6. FIG. 9 is a conceptual diagram corresponding to the functional configuration in FIG. 8.

In the excrement management system 1 illustrated in FIG. 4 and FIG. 5, the closet bowl device 2 and the detection device 12 are configured to be separated from each other. The detection device 12 is disposed in the toilet room R by being hung between the rim part 9 of the toilet bowl 7 and the toilet seat 5, for example.

In the excrement management system 1 illustrated in FIG. 4 and FIG. 5, the user identification device 38 is disposed in the toilet room R, and configured to be able to communicate with the closet bowl device 2. Identification of the user by the user identification device 38 is, for example, implemented by an input from the user to the operation device 10 disposed in the toilet room R, various sensors disposed in the toilet room R, and the like.

In the excrement management system 1 illustrated in FIG. 4 and FIG. 5, the edge server 34 and the cloud server 30 are disposed outside the toilet room R.

In the excrement management system 1 illustrated in FIG. 4 and FIG. 5, the closet bowl device 2 is configured to be able to communicate with the edge server 34 via the second communication device 36, and the edge server 34 is configured to be able to communicate with the cloud server 30 via the first communication device 32.

In the excrement management system 1 illustrated in FIG. 4 and FIG. 5, the user information identified by the user identification device 38 and the data detected by the detection device 12 are processed by the control device 20 included in the closet bowl device 2, and transmitted to the edge server 34 via the second communication device 36. By analyzing the detection data by the edge server 34, it is determined whether to transmit the detection data to the cloud server 30 by the first communication device 32. Due to this, it is possible to prevent all pieces of the data detected by the detection device 12 from being entirely transmitted to the cloud server 30, so that data communication amount to the cloud server 30 can be suppressed.

In the excrement management system 1 illustrated in FIG. 4 and FIG. 5, a plurality of the closet bowl devices 2 can be connected to one edge server 34. Due to this, the number of the edge servers 34 required for the excrement management system 1 can be reduced, so that cost required for constructing the excrement management system 1 can be reduced.

Furthermore, in the excrement management system 1 illustrated in FIG. 4 and FIG. 5, the user information transmitted to the edge server 34 is anonymized by the edge server 34 so that an individual cannot be specified, and transmitted to the cloud server 30 together with the detection data via the first communication device 32 thereafter. Due to this, the user information is excluded from the light reception data transmitted from the edge server, so that communication capacity from the edge server 34 to the cloud server 30 can be reduced. Additionally, individual information can be prevented from leaking out in the communication path.

The determination result related to the characteristic amount of the excrement analyzed by the cloud server 30 is transmitted to the edge server 34 via the first communication device 32, and associated with the user information by the edge server 34 to be displayed on a display device (not illustrated) disposed in the toilet room R in which the user identification device 38 is disposed. Due to this, the user of the toilet room R can confirm whether the determination result displayed on the display device is a result of himself/herself. A destination of the determination result sent by the edge server 34 is not limited to the toilet room R, and the determination result may be sent to the portable terminal and the like of the user that is previously stored in the edge server 34.

In the excrement management system 1 illustrated in FIG. 6 and FIG. 7, functions of the detection device 12 and the edge server 34 are implemented by the closet bowl device 2. The detection device 12 is, for example, disposed inside the functional unit 6 or the toilet seat 5 included in the toilet seat device 3. The function of the edge server 34 is implemented by the control device 20 included in the closet bowl device 2.

In the excrement management system 1 illustrated in FIG. 6 and FIG. 7, the function of the user identification device 38 is implemented by a portable terminal 40 used by a care worker and the like of a nursing home. The portable terminal 40 is, for example, constituted of a smartphone, a mobile phone, a tablet terminal, and the like. The portable terminal 40 may be carried by a care worker and the like, or may be disposed outside the toilet room R. Identification of the user by the portable terminal 40 is implemented by a user ID and the like for identifying the user.

In the excrement management system 1 illustrated in FIG. 6 and FIG. 7, the cloud server 30 is disposed outside the toilet room R.

In the excrement management system 1 illustrated in FIG. 6 and FIG. 7, the closet bowl device 2 is configured to be able to communicate with the cloud server 30 via the first communication device 32. The detection device 12 and the portable terminal 40 are configured to be able to communicate with the edge server 34 via the second communication device 36.

In the excrement management system 1 illustrated in FIG. 6 and FIG. 7, the data detected by the detection device 12 is transmitted to the control device 20 by the second communication device 36 that is configured by serial communication and the like. The user information identified by the portable terminal 40 is transmitted to the control device 20 by the second communication device 36 configured by short-range wireless communication and the like. By analyzing the detection data by the control device 20, it is determined whether to transmit the detection data to the cloud server 30 by the first communication device 32. Due to this, it is possible to prevent all pieces of the data detected by the detection device 12 from being entirely transmitted to the cloud server 30, so that the data communication amount to the cloud server 30 can be suppressed.

In the excrement management system 1 illustrated in FIG. 6 and FIG. 7, the determination result related to the characteristic amount of the excrement analyzed by the cloud server 30 is enabled to be accessed from the portable terminal 40 of the user via the first communication device 32 or the second communication device 36. Due to this, the determination result related to the characteristic amount of the excrement can be confirmed from the portable terminal 40 of the user. The user information is stored in the portable terminal 40 of the user, so that the cloud server 30 does not necessarily manage the user information.

In the excrement management system 1 illustrated in FIG. 8 and FIG. 9, the function of the detection device 12 is implemented by the closet bowl device 2.

In the excrement management system 1 illustrated in FIG. 8 and FIG. 9, the functions of the edge server 34 and the user identification device 38 are implemented by the portable terminal 40. The function of the edge server 34 is implemented by the control device 20 included in the portable terminal 40. In this case, a computer program with which the portable terminal 40 implements the function of the edge server 34 is downloaded from the cloud server 30 in a format of application software, for example.

In the excrement management system 1 illustrated in FIG. 8 and FIG. 9, the cloud server is disposed outside the toilet room R.

In the excrement management system 1 illustrated in FIG. 8 and FIG. 9, the portable terminal 40 of the user is configured to be able to communicate with the closet bowl device 2 via the second communication device. The portable terminal 40 of the user is also configured to be able to communicate with the cloud server 30 via the first communication device.

In the excrement management system 1 illustrated in FIG. 8 and FIG. 9, the data detected by the detection device 12 is transmitted to the portable terminal 40 of the user via the second communication device 36 configured by short-range wireless communication and the like, and processed by the control device 20 included in the portable terminal 40 together with the user information stored in the portable terminal 40. By analyzing the detection data by the portable terminal 40, it is determined whether to transmit the detection data to the cloud server 30 by the first communication device 32. Due to this, it is possible to prevent all pieces of the data detected by the detection device 12 from being entirely transmitted to the cloud server 30, so that the data communication amount to the cloud server 30 can be suppressed.

In the excrement management system 1 illustrated in FIG. 8 and FIG. 9, the data detected by the detection device 12 can be processed by the control device 20 included in the closet bowl device 2 to reduce the data amount before being transmitted to the portable terminal 40. Due to this, data transfer speed to the portable terminal via the second communication device 36 can be increased. Furthermore, in the excrement management system 1 illustrated in FIG. 6, the control device 20 included in the portable terminal 40 can perform simpler analysis than the analysis related to the characteristic amount of the excrement performed by the cloud server 30. For example, by performing analysis related to only presence/absence of the excrement, an owner of the portable terminal 40 can grasp an excretion period of himself/herself without transmitting the detection data to the cloud server 30 via the first communication device 32.

Furthermore, in the excrement management system 1 illustrated in FIG. 8 and FIG. 9, the determination result related to the characteristic amount of the excrement analyzed by the cloud server 30 can be enabled to be accessed from the portable terminal 40 of the user via the first communication device 32. Due to this, the determination result related to the characteristic amount of the excrement can be confirmed from the portable terminal 40 of the user. The user information is stored in the portable terminal 40 of the user, so that the cloud server 30 does not necessarily manage the user information.

4. Processing of Excrement Management System

Next, the following describes processing for the information about the excrement collected by the excrement management system 1.

4-1. Data

First, the following describes the data detected by the detection device 12 with reference to FIG. 10. FIG. 10 is a diagram illustrating an example of processing for the data detected by the detection device. The following describes only configurations and processing required for a data flow, and light emission from the light emitting unit and the like will not be described.

First, the light receiving element of the light receiving unit 16 performs detection. The light receiving unit detects analog data AD1 of N pixels (N is an optional number). The analog data AD1 detected by the light receiving unit 16 is transmitted to the control device 20 included in the closet bowl device 2 or the detection device 12 (Step S11).

The control device 20 includes an AD Converter, and converts the analog data AD1 of an analog value into digital data of a digital value. The control device 20 determines a pixel to be AD-converted by the AD Converter, and determines a pixel to be converted by the AD Converter in the analog data AD1 of N pixels. The control device 20 determines a value “n” equal to or smaller than N, and determines the number of pixels “n” to be converted by the AD Converter. For example, by determining the value equal to or smaller than N to be “n”, the control device 20 can reduce a data amount to be transmitted to the cloud server 30 later.

The control device 20 temporarily stores AD-converted digital data DD1 in the memory 22 included in the control device 20 (Step S12). In accordance with control by the control device 20, digital data of n pixels is stored in a storage region FM1 of the memory 22 included in the control device 20.

When the amount of the digital data stored in the storage region FM1 becomes equal to or larger than a predetermined amount including n pixels×m rows, the digital data of n pixels×m rows is transmitted to the edge server 34 via the second communication device 36 included in the closet bowl device 2 or the detection device 12 (Step S13). In a case in which the function of the edge server 34 is executed by the closet bowl device 2, excretion determination (described later) is performed on the digital data of n pixels×m rows by the arithmetic processing device 24 included in the control device 20.

The edge server 34 performs excretion determination on the digital data of n pixels×m rows transmitted from the control device 20 for determining whether the excrement is included therein. For example, the edge server 34 performs threshold determination on predetermined n-l pixels×m rows in the digital data of n pixels. The edge server 34 may perform threshold determination on the digital data of n pixels×m rows.

In accordance with a result of threshold determination, the edge server 34 determines whether to transmit the digital data transmitted from the control device 20 to the cloud server 30 via the first communication device 32. In other words, the edge server 34 determines whether to transmit the data to the cloud server 30 by a device that executes the function of the first communication device 32.

As a result of excretion determination by the edge server 34, in a case in which the number of pixels having the light receiving element the output value of which varies from initial data by a predetermined value or more is smaller than a threshold, the digital data transmitted from the control device 20 is deleted as illustrated in a storage region FM2 (Step S14). That is, in a case of determining that the light reception data received by the light receiving element is not light reception data reflected by the excrement, the edge server 34 deletes the digital data (for example, the digital data of n pixels×m rows) stored in the storage region FM2 included in the edge server 34. Alternatively, in a case in which data that is continuously received by the light receiving element, for example, image data and the like, does not change, the image data may be deleted.

In this way, in a case of determining that the data transmitted via the second communication device 36 is not the light reception data reflected from the excrement, the edge server 34 does not transmit the digital data to the cloud server 30 via the first communication device 32. That is, the data is not allowed to be transmitted to the cloud server 30 by the device that controls the first communication device 32.

As a result of excretion determination by the edge server 34, in a case in which the number of pixels having the light receiving element the output value of which varies from the initial data by the predetermined value or more is equal to or larger than the threshold, the data transmitted from the control device 20 is transmitted to the cloud server 30 via the first communication device 32 as illustrated in a storage region FM3 (S15). That is, in a case of determining that the light reception data received by the light receiving element is the light reception data reflected by the excrement, the edge server 34 transmits the digital data (for example, the digital data of n pixels×m rows) stored in the storage region FM2 included in the edge server 34 to the cloud server 30 via the first communication device 32. For example, by causing the data that is transmitted via the first communication device 32 and stored in the storage region FM3 included in the cloud server 30 to be data constituted of only the number of pixels (for example, n-l pixels×m columns) having the light receiving element the output value of which varies from the initial data by the predetermined value or more as a result of excretion determination by the edge server 34, the data amount transmitted to the cloud server 30 can be reduced.

In this way, in a case of determining that the data transmitted via the second communication device 36 is the light reception data reflected from the excrement, the edge server 34 transmits the digital data to the cloud server 30 via the first communication device 32. That is, the data is allowed to be transmitted to the cloud server 30 by the device that controls the first communication device 32.

The cloud server 30 performs determination about the characteristic amount of the excrement on the digital data transmitted via the first communication device 32. The cloud server 30 then stores a result thereof in the storage region FM3 included in the cloud server 30 (S16). The determination result obtained by the cloud server 30 may be transmitted to the edge server 34 via the first communication device 32 without being stored in the cloud server 30.

For example, the cloud server 30 performs determination about the three characteristic amounts including the color, the shape, and the amount of the excrement. The cloud server 30 stores the determination result for the color of the excrement using 3 bits of the storage region FM3 so that eight types (yellow, brown, black, abnormal (including red of blood), and the like) can be determined at the maximum. The cloud server stores the determination result for the shape of the excrement using 3 bits of the storage region FM3 so that seven types of Bristol stool form scale can be determined. The cloud server 30 stores the determination result for the amount of the excrement using 2 bits of the storage region FM3 so that at least three types including large, normal, and small can be determined. Due to this, the cloud server 30 can store the determination result for the three characteristic amounts including the color, the shape, and the amount of the excrement using 1 byte of the storage region FM3. Processing for determining the characteristic amount of the excrement by the cloud server 30 will be described later.

4-2. Data Analysis

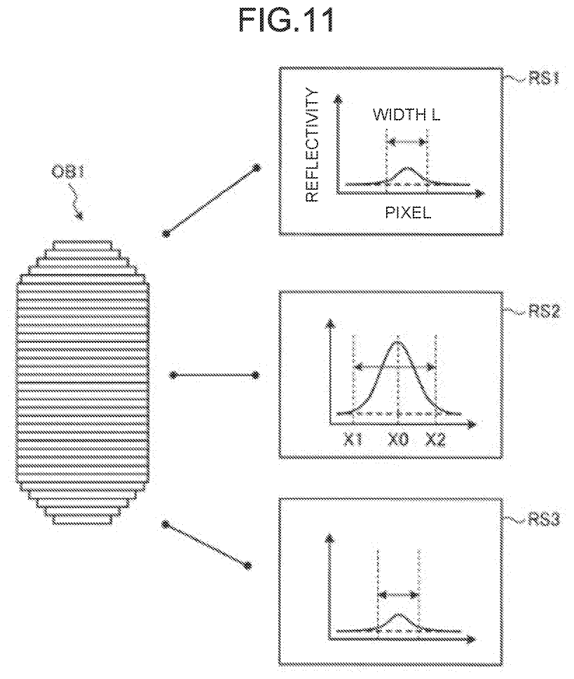

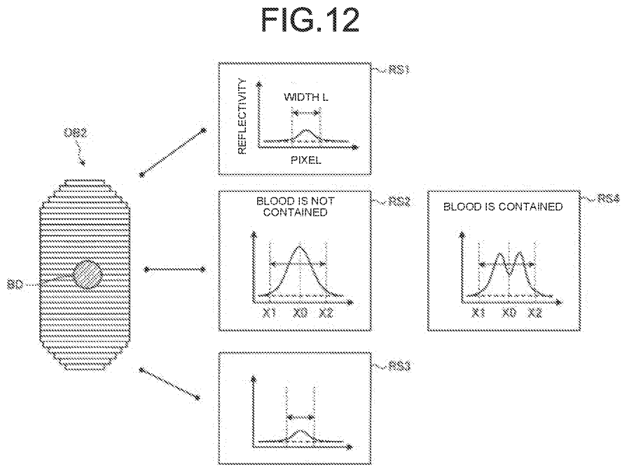

The following describes data analysis for determining the characteristic amount of the excrement with reference to FIG. 11 and FIG. 12. The following describes processing of performing data analysis related to the color, the shape, and the amount of the excrement by the cloud server 30 of the excrement management system 1.

4-2-1. Shape and Amount of Excrement

First, the following describes data analysis related to the shape and the amount of the excrement with reference to FIG. 11. FIG. 11 is a diagram illustrating an example of data analysis related to the shape and the amount of the excrement.

An object OB1 in FIG. 11 schematically represents excrement (feces) as a detection target, and the following describes an outline of how the shape and the amount of the excrement is analyzed using the object OB1 as an example. In the following description, a longitudinal direction of the object OB1 is assumed to be a vertical direction, and a direction orthogonal to the longitudinal direction (lateral direction) is assumed to be a horizontal direction. The object OB1 falls in a direction along the vertical direction.

Each of measurement results RS1 to RS3 is a graph representing a relation between each pixel and reflectivity thereof. Each of the measurement results RS1 to RS3 indicates a measurement result corresponding to each position in the vertical direction of the object OB1. The measurement result RS1 indicates a measurement result corresponding to an upper end of the object OB1. The measurement result RS2 indicates a measurement result corresponding to a center part in the vertical direction of the object OB1. The measurement result RS3 indicates a measurement result corresponding to a lower end of the object OB1.

The cloud server 30 detects reflectivity of each pixel received by the light receiving element. The cloud server 30 obtains a peak value from among pixels that have caused reflection. A center portion of each of the measurement results RS1 to RS3 is the peak value. For example, the cloud server 30 specifies that a pixel X0 has a peak value in the image of the measurement result RS2.

In a case in which a difference in reflectivity between the pixel having the peak value and a pixel adjacent thereto is compared and reflectivity equal to or larger than a predetermined value or equal to or smaller than the predetermined value is confirmed, the cloud server 30 estimates that there is reflected light from the excrement. The cloud server 30 performs processing for the color of the excrement in a similar way.

In a case in which the reflected light from the excrement is confirmed, the cloud server 30 further performs similar processing on a pixel adjacent to the former pixel. Due to this, the cloud server 30 grasps an end of the excrement, and estimates the width of the excrement. For example, in the measurement result RS2, the cloud server 30 estimates that a range from a pixel X1 to an image X2 is the excrement. For example, in the measurement result RS1, the cloud server 30 estimates that a width L narrower than the range from the pixel X1 to the image X2 in the measurement result RS2 is the width of the excrement.

The cloud server 30 stacks the measurement results RS1 to RS3 and the like to analyze the shape of the excrement. In the example of FIG. 11, the cloud server 30 analyzes the shape such that the portion corresponding to the measurement result RS2 (center part) has the largest width, and the width is reduced as being closer to the portion corresponding to the measurement result RS1 (upper end part) and the portion corresponding to the measurement result RS3 (lower end part). The cloud server 30 determines which one of the seven types of shapes of excrement (feces) classified by Bristol stool form scale is the closest to the shape of the excrement that is analyzed based on the measurement result, and determines the shape of the excrement excreted by the user.

The cloud server 30 adds up the number of pixels that are estimated to be reflected light from the excrement to analyze the amount of the excrement. In a case in which there are a plurality of pieces of excrement (feces) excreted by the user, the amount of the excrement that is excreted through one time of excretion action performed by the user is analyzed by adding up the amount of the pieces of excrement.

Through the processing described above, the object OB1 falling from the user toward the bowl part 8 of the toilet bowl 7 is detected. For example, the object OB1 as falling excrement passes through the lower end part, the center part, and the upper end part in this order in front of the light emitting unit 14 and the light receiving unit 16, and is detected in order from a lower part to an upper part thereof. Specifically, the object OB1 as falling excrement is detected in order of the measurement result RS3, the measurement result RS2, and the measurement result RS1. The excrement analyzed by the cloud server 30 is not limited to falling excrement, and detection may be performed on excrement that has landed on water in the bowl part 8 after falling.

4-2-2. Color of Excrement

First, the following describes data analysis related to the color of the excrement with reference to FIG. 12. FIG. 12 is a diagram illustrating an example of data analysis of the color of the excrement. FIG. 12 is a diagram illustrating an example of data analysis related to detection of blood contained in the excrement. The same points as those in FIG. 11 are denoted by the same reference numerals, and the description thereof will be omitted as appropriate.

An object OB2 in FIG. 12 represents virtual excrement (feces), and is different from the object OB1 in FIG. 11 in that a blood region BD is included in the center part of the object OB2. The measurement results RS1 to RS3 illustrated in FIG. 12 correspond to the measurement results RS1 to RS3 of the object OB1 in FIG. 11, which does not include the blood region BD.

The cloud server 30 specifies a pixel that has a peak value with respect to light of a wavelength having characteristic reflectivity for blood among pieces of light of a plurality of wavelengths emitted to the object OB2 as excrement. For example, the cloud server 30 specifies a pixel that has a peak value with respect to light of 670 nm having characteristic reflectivity for blood among the pieces of light of a plurality of wavelengths emitted to the object OB2 as excrement.

Thereafter, the cloud server 30 calculates reflectivity with respect to light of the other wavelength detected by the pixel having the peak value. The cloud server 30 estimates the color based on a ratio of reflectivity with respect to the other wavelength including 670 nm detected by the above pixel. A measurement result RS4 illustrated in FIG. 12 indicates a measurement result for a point including the blood region BD such as the object OB2. For example, the measurement result RS4 illustrated in FIG. 12 indicates a measurement result in a case in which light of a region not including 670 nm is emitted to the portion including the blood region BD of the object OB2.

The wavelength having characteristic reflectivity for blood is not limited to 670 nm, and may be in a range from 600 nm to 800 nm. This is because, in a case in which blood adheres to the feces, reflectivity for the color of the blood is detected more clearly than that for the color of the feces in this wavelength band.

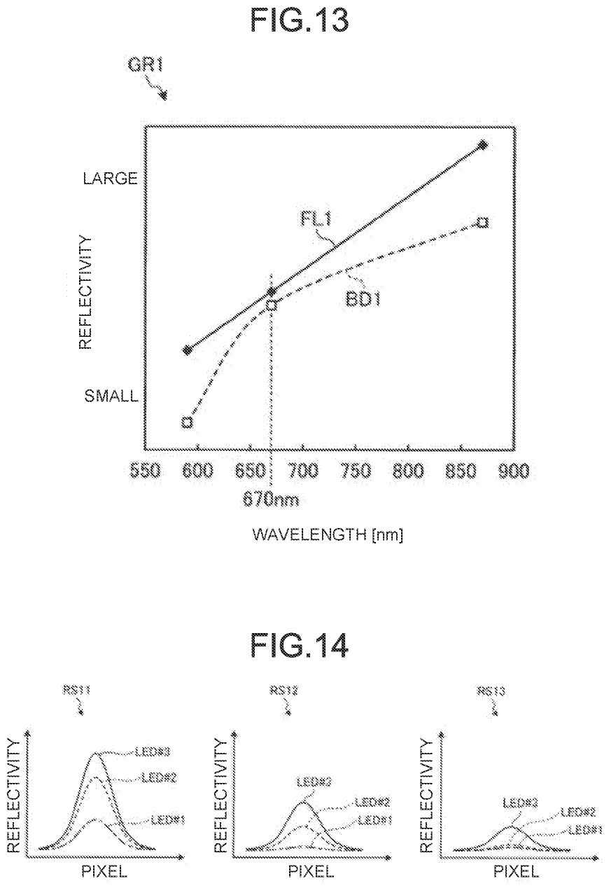

The following describes a relation between the excrement and the blood with reference to FIG. 13. FIG. 13 is a diagram illustrating an example of the relation between the excrement and the blood. A graph GR1 illustrated in FIG. 13 represents a relation between reflection of feces and reflection of blood adhering to the feces with respect to each wavelength.

A line FL1 in the graph GR1 of FIG. 13 indicates reflectivity at each wavelength (about 600 nm to about 870 nm) with respect to the excrement (feces). As indicated by the line FL1 in FIG. 13, in a case of the excrement (feces), the reflectivity is increased as the wavelength becomes longer. As indicated by the line FL1 in FIG. 13, in a case of the excrement (feces), the reflectivity around 600 nm is the lowest, and the reflectivity around 870 nm is the highest. A line BD1 in the graph GR1 of FIG. 10 indicates the reflectivity at each wavelength (about 600 nm to about 870 nm) with respect to blood adhering to the feces. As indicated by the line BD1 in FIG. 13, in a case of blood adhering to the feces, a difference between the reflectivity around 670 nm and the line FL1 is the smallest, and the difference between the reflectivity and the line FL1 becomes larger as being distant from 670 nm.

In the graph GR1 in FIG. 13, a ratio of the reflectivity of blood adhering to the feces with respect to the reflectivity of the feces is the largest around 670 nm, and becomes smaller as being distant from 670 nm. In this way, in the graph GR1 illustrated in FIG. 13, the ratio of the reflectivity of blood adhering to the feces with respect to the reflectivity of the feces is large at the wavelength of 670 nm, and the ratio of the reflectivity of blood with respect to the reflectivity of the feces is small at the wavelength of 870 nm.

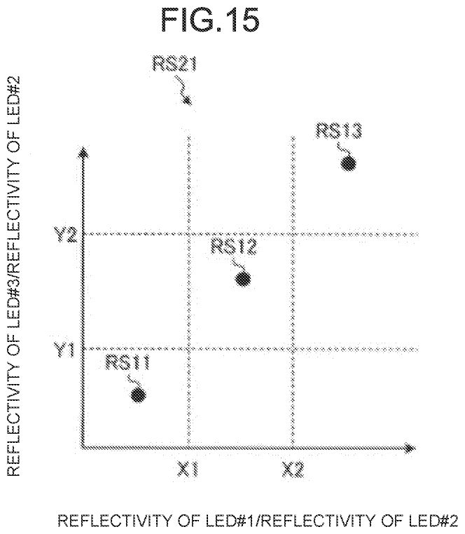

Thus, the cloud server 30 can analyze the blood contained in the excrement based on the ratio of the reflectivity at each wavelength as described above. The cloud server 30 can also analyze the color of the excrement based on the ratio of the reflectivity at each wavelength as described above. This point will be described below with reference to FIG. 14 and FIG. 15. FIG. 14 and FIG. 15 are diagrams illustrating examples of data analysis of the color of the excrement.

Measurement results RS11 to RS13 illustrated in FIG. 14 indicate measurement results in a case in which pieces of the excrement (feces) having different colors are measurement targets. For example, the color of the excrement (feces) as the measurement target may become darker in order of the measurement results RS11, RS12, and RS13. For example, the measurement result RS11 may be a measurement result of ocherous excrement (feces), the measurement result RS12 may be a measurement result of brown excrement (feces), and the measurement result RS13 may be a measurement result of dark-brown excrement (feces).

Each of LED#1, LED#2, and LED#3 indicated by the respective measurement results RS11 to RS13 in FIG. 14 is a light emitting element that emits light, and each curved line of LED#1, LED#2, and LED#3 indicates a relation between the pixel and the reflectivity. Each of LED#1, LED#2, and LED#3 may be a light emitting element that emits light in any wavelength region.

For example, the reflectivity with respect to each wavelength becomes smaller as the color of the feces is darker. In the example of FIG. 14, the reflectivity with respect to each wavelength becomes small in the measurement result RS13 in which the color of the excrement (feces) is the darkest among the measurement results RS11 to RS13, and the ratio of reflectivity therebetween becomes large.

On the other hand, for example, the reflectivity with respect to each wavelength becomes larger as the color of the feces is lighter. In the example of FIG. 14, the reflectivity with respect to each wavelength becomes large in the measurement result RS11 in which the color of the excrement (feces) is the lightest among the measurement results RS11 to RS13, and the ratio of reflectivity therebetween becomes small. For example, light of each wavelength is reflected more strongly as being closer to a lighter color, so that a difference in reflectivity at each wavelength becomes smaller.

Thus, the cloud server 30 can classify the color of the excrement (feces) by performing analysis based on the relation between the wavelength and the reflectivity as described above. For example, as in a classification result RS21 illustrated in FIG. 15, the cloud server 30 classifies the color of the excrement (feces) in each measurement by classifying the measurement results RS11 to RS13 based on the ratio of reflectivity with respect to each of LED#1, LED#2, and LED#3.

For example, the cloud server 30 classifies the color of the excrement (feces) in each of the measurement results RS11 to RS13 using a ratio between the reflectivity of LED#1 and the reflectivity of LED#2, or a ratio between the reflectivity of LED#3 and the reflectivity of LED#2. For example, assuming that an X-axis indicates “reflectivity of LED#1/reflectivity of LED#2” and a Y-axis indicates “reflectivity of LED#3/reflectivity of LED#2”, the cloud server 30 classifies the color of the excrement (feces) in each measurement in accordance with a position of each of the measurement results RS11 to RS13. For example, in a case in which the position is smaller than X1 in the X-axis direction and smaller than Y1 in the Y-axis direction, the cloud server 30 classifies the color of the excrement (feces) in that measurement as “ocher”. For example, in a case in which the position is equal to or larger than X1 and smaller than X2 in the X-axis direction, and equal to or larger than Y1 and smaller than Y2 in the Y-axis direction, the cloud server 30 classifies the color of the excrement (feces) in that measurement as “brown”. For example, in a case in which the position is equal to or larger than X2 in the X-axis direction, and equal to or larger than Y2 in the Y-axis direction, the cloud server 30 classifies the color of the excrement (feces) in that measurement as “dark brown”. These are merely examples, and the cloud server 30 may classify the color of the excrement (feces) in each measurement using any method.

### REFERENCE SIGNS LIST

1 EXCREMENT MANAGEMENT SYSTEM

2 CLOSET BOWL DEVICE

3 TOILET SEAT DEVICE

4 TOILET LID

5 TOILET SEAT

6 FUNCTIONAL UNIT

7 TOILET BOWL

8 BOWL PART

9 RIM PART

10 OPERATION DEVICE

12 DETECTION DEVICE

14 LIGHT EMITTING UNIT

16 LIGHT RECEIVING UNIT

17 LENS

18 HOUSING

20 CONTROL DEVICE

22 MEMORY

24 ARITHMETIC PROCESSING DEVICE

26 ELECTRONIC CIRCUIT

30 CLOUD SERVER

32 FIRST COMMUNICATION DEVICE

34 EDGE SERVER

36 SECOND COMMUNICATION DEVICE

38 USER IDENTIFICATION DEVICE

40 PORTABLE TERMINAL

What is claim is:

1. An excrement management system configured to collect and manage information about excrement, the excrement management system comprising:
a closet bowl in which a bowl part for receiving excrement is formed;
a light emitting unit configured to emit light toward an inner part of the closet bowl;
a light receiving unit comprising an image sensor configured to receive light;
a cloud server and an edge server configured to analyze light reception data received by the light receiving unit;
a first communication device configured to transmit the light reception data to the cloud server; and
a second communication device configured to transmit the light reception data to the edge server, wherein
the cloud server analyzes the light reception data to determine a characteristic of excrement, and
the edge server analyzes the light reception data to determine whether to transmit the light reception data to the cloud server by the first communication device.

2. The excrement management system according to claim 1, wherein the first communication device transmits the light reception data to the cloud server by using a wide-area information communication network, and the second communication device transmits the light reception data to the edge server by using a local-area information communication network.

3. The excrement management system according to claim 2, wherein
the first communication device is configured to perform transmission/reception of data between the cloud server and the edge server, and
data capacity that is transmitted from the cloud server to the edge server via the first communication device is smaller than data capacity of the light reception data that is transmitted from the edge server to the cloud server via the first communication device.

4. The excrement management system according to any claim 1, wherein
the cloud server analyzes the light reception data to determine at least one of three characteristic amounts including a color, a shape, and an amount of excrement, and
the edge server analyzes the light reception data to determine whether excrement is included in the light reception data.

5. The excrement management system according to claim 1, further comprising:
a user identification device configured to acquire user information who uses the closet bowl, wherein
the first communication device does not transmit the user information to the cloud server.

6. The excrement management system according to claim 1, wherein
user information about a user who uses the closet bowl is previously recorded in the cloud server, and
the light reception data that is determined to be transmitted by the edge server is associated with the user information that is previously recorded in the cloud server.

7. An excretion information management method for managing, on a cloud server, information about excrement collected in a toilet room in which a closet bowl is disposed, the excretion information management method comprising:
a detection step of receiving, by a light receiving unit, reflected light from excrement corresponding to light that is emitted toward an inner part of the closet bowl by a light emitting unit; and
an analysis step of determining whether to transmit the light reception data to the cloud server by a communication device based on light reception data detected at the detection step.

8. A computer-readable recording medium having stored a computer program executed by an edge server configured to be able to communicate with a closet bowl device and a cloud server, the computer program configured to cause an edge server to perform:
a reception procedure of receiving light reception data that is detected by receiving, by a light receiving unit, reflected light from excrement corresponding to light that is emitted toward an inner part of a closet bowl by a light emitting unit; and
a transmission procedure of transmitting a determination result about whether to transmit the light reception data to a device configured to control a communication device that transmits the light reception data to the cloud server based on an analysis result about the light reception data.

9. An edge server configured to be able to communicate with a cloud server and a closet bowl device, the edge server comprising:
a first communication device configured to be able to communicate with the cloud server;
a second communication device configured to be able to communicate with the closet bowl device;
a memory configured to store detection data related to excrement that is optically detected and transmitted from the closet bowl device via the second communication device; and
an arithmetic processing device configured to analyze the detection data stored in the memory, wherein
the arithmetic processing device determines whether to transmit the detection data to the cloud server by the first communication device based on the detection data.

10. A toilet seat device disposed on an upper part of a closet bowl, the toilet seat device comprising:
a light emitting unit configured to emit light toward an inner part of the closet bowl;
a light receiving unit comprising an image sensor configured to receive light;
a memory configured to store light reception data received by the light receiving unit;
a communication device configured to transmit the light reception data to a cloud server; and
an arithmetic processing device configured to analyze the light reception data stored in the memory, wherein
the arithmetic processing device determines whether to transmit the light reception data to the cloud server based on the light reception data.
